(19) United States
(12) Patent Application Publication
(10) Pub. No.: US 2005/0214343 A1
(43) Pub. Date: Sep. 29, 2005

(54) Medical devices comprising a protein-tyrosine kinase inhibitor to inhibit restonosis

(76) Inventors: Patrice Tremble, Santa Rosa, CA (US); Wenda Carlyle, Silverado, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/490,248

(22) PCT Filed: Jul. 17, 2003
(86) PCT No.: PCT/US03/22546

§ 371 Date: Mar. 14, 2005

Related U.S. Application Data
(60) Provisional application No. 60/397,149, filed on Jul. 18, 2002.

(51) Int. Cl. A61K 31/506; A61F 2/00

(52) U.S. Cl. 424/426; 514/252.18

(57) ABSTRACT

Implantable medical devices having an anti-restenotic coatings are disclosed. Specifically, implantable medical devices having coatings of proteintyrosine kinase inhibitors are disclosed. The anti-restenotic protein-tyrosine kinase inhibitor is 4+4-Methyl-1-piperazinyl)methyl]-N-[4-methyl-3-[[4-(3-pyridinyl)-2pyrimidinyl]amino]-phenyl]benzamide methanesulfonate and pharmaceutically acceptable derivatives thereof (imatinib mesylate). The anti-restenotic medial devices include stents, catheters, micro-particles, probes and vascular grafts. The medical devices can be coated using any method known in the art including compounding the protein-tyrosine kinase inhibitor with a biocompatible polymer prior to applying the coating. Moreover, medical devices composed entirely of biocompatible polymer-protein-tyrosine kinase inhibitor blends are disclosed. Additionally, medical devices having a coating comprising at least one proteintyrosine kinase inhibitor in combination with at least one additional therapeutic agent are also disclosed. Furthermore, related methods of using and making the antirestenotic implantable devices are also disclosed.

### FIELD OF THE INVENTION

The present invention relates to medical devices and methods of using medical devices to treat or inhibit restenosis. Specifically, the present invention relates to stents that provide in situ controlled release delivery of anti-restenotic compounds. More specifically, the present invention provides vascular stents that provide anti-restenotic effective amounts of imatinib mesylate directly to tissues at risk for restenosis.

### BACKGROUND OF THE INVENTION

Cardiovascular disease, specifically atherosclerosis, remains a leading cause of death in developed countries. Athersclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMC proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is a percutaneous procedure wherein a balloon catheter is inserted into the coronary artery and advanced until the vascular stenosis is reached. The balloon is then inflated restoring arterial patency. One angioplasty variation includes arterial stent deployment. Briefly, after arterial patency has been restored, the balloon is deflated and a vascular stent is inserted into the vessel lumen at the stenosis site. The catheter is then removed from the coronary artery and the deployed stent remains implanted to prevent the newly opened artery from constricting spontaneously. However, balloon catheterization and stent deployment can result in vascular injury ultimately leading to VSMC proliferation and neointimal formation within the previously opened artery. This biological process whereby a previously opened artery becomes re-occluded is referred to as restenosis.

Treating restenosis requires additional, generally more invasive, procedures including CABG in severe cases. Consequently, methods for preventing restenosis, or treating incipient forms, are being aggressively pursued. One possible method for preventing restenosis is the administration of anti-inflammatory compounds that block local invasion/activation of monocytes thus preventing the secretion of growth factors that may trigger VSMC proliferation and migration. Other potentially anti-restenotic compounds include anti-proliferative agents such as chemotherapeutics including rapamycin and paclitaxel. Rapamycin is generally considered an immunosuppressant best known as a organ transplant rejection inhibitor. However, rapamycin is also used to treat severe yeast infections and certain forms of cancer. Paclitaxel, known by its trade name Taxol®, is used to treat a variety of cancers, most notably breast cancer.

However, anti-inflammatory and anti-proliferative compounds can be toxic when administer systemically in anti-restenotic-effective amounts. Furthermore, the exact cellular functions that must be inhibited and the duration of inhibition needed to achieve prolonged vascular patency (greater than six months) is not presently known. Moreover, it is believed that each drug may require its own treatment duration and delivery rate. Therefore, in situ, or site-specific drug delivery using anti-restenotic coated stents has become the focus of intense clinical investigation.

Recent human clinical studies on stent-based anti-restenotic delivery have centered on rapamycin and paclitaxel. In both cases excellent short-term anti-restenotic effectiveness has been demonstrated. However, side effects including vascular erosion have also been seen. Vascular erosion can lead to stent instability and further vascular injury. Furthermore, the extent of cellular inhibition may be so extensive that normal re-endothelialization will not occur. The endothelial lining is essential for maintaining vascular elasticity and as an endogenous source of nitric oxide. Therefore, compounds that exert localized anti-restenotic effects while minimizing vascular and cellular damage are essential for the long-term success of drug delivery stents.

### SUMMARY OF THE INVENTION

The present invention provides an in situ drug delivery platform that can be used to administer anti-restenotic tissue levels of protein-tyrosine kinase inhibitors without systemic side effects. In one embodiment of the present invention the drug delivery platform is a medical device including, without limitations, stents, catheters, micro-particles, probes and vascular grafts.

In another embodiment of the present invention, a vascular stent is coated with imatinib mesylate. The imatinib mesylate can be attached to the vascular stent's surface using any means that provide a drug-releasing platform. Coating methods include, but are not limited to precipitation, coacervation, and crystallization. The imatinib mesylate of the present invention can be bound covalently, ionically, or through other intramolecular interactions including without limitation hydrogen bonding and van der Waals forces.

In another embodiment of the present invention the imatinib mesylate is complexed with a suitable biocompatible polymer. The polymer-drug complex is then used to either form a controlled-release medical device, integrated into a preformed medical device or used to coat a medical device. The biocompatible polymer may be any non-thrombogenic material that does not cause a clinically relevant adverse response. Other methods of achieving controlled drug release are contemplated as being part of the present invention.

Moreover, the imatinib mesylate of the present invention can be combined with other anti-restenotic compounds including cytotoxic, cytostatic, anti-metabolic and anti-inflammatory compounds.

In yet another embodiment of the present invention an anti-restenotic compound coated stent can be combined with the systemic delivery of the same or another anti-restenotic compound to achieve a synergistic or additive effect at the medical device placement site. This is particularly beneficial in that non-toxic therapeutic levels of both imatinib mesylate and other anti-restenotic therapeutics can be combined to achieve dose specific synergism.

In one embodiment of the present invention the imatinib mesylate is directly coated onto the surface of a metal stent.

In another embodiment of the present invention the stent is coated with a bioerodable polymer having the imatinib mesylate dispersed therein.

In another embodiment of the present invention the stent is coated with a non-bioerodable polymer having the imatinib mesylate dispersed therein.

In yet another embodiment of the present invention a stent is coated with a first polymer layer having the imatinib mesylate dispersed therein and a second layer of polymer provided over the first polymer layer.

In yet another embodiment of the present invention a stent is provided with an imatinib mesylate coating and at least one other antiplatelet, antimigratory, antifibrotic antiproliferatives and/or antiinflammatories agent combined therewith.

In yet another embodiment of the present invention the stent is selected from the group consisting of vascular stents, biliary stents, esophageal stent, and urethral stents.

In still another embodiment of the present invention the stent is a polymer stent.

Additional embodiments of the present invention will be apparent to those skilled in the art from the drawings and detailed disclosure that follows.

### BRIEF DESCRIPTION OF THE DRAWINGS

FIG. 1 depicts a vascular stent used to deliver the antirestenotic compounds of the present invention.

FIG. 2 depicts a balloon catheter assembly used for angioplasty and the site specific delivery of stents to anatomical lumens at risk for restenosis.

FIGS. 3 and 4 depict various coating configurations for a vascular stent coated in accordance with the teachings of the present invention.

### DETAILED DESCRIPTION OF THE PRESENT INVENTION

The present invention relates to restoring patency to anatomical lumens that have been occluded, or stenosed, as a result of mechanical trauma, surgical injury, pathologies or normal biological processes including genetic anomalies. The present invention can be used to restore and maintain patency in any anatomical lumen, including, but not limited to blood vessels, ducts such as the biliary duct, and wider lumens including the esophagus and urethra. Furthermore, graft site associated stenoses can also be treated using the teachings of the present invention.

In one embodiment of the present invention the stenosed lumen is an artery, specially a coronary artery. Stenosed coronary arteries generally result from plaque that develops on the interior lining of a coronary artery. Present models attribute this pathology to vascular injuries that are associated with life style and diet. Two major categories of vascular plaque are thought to contribute to over 90% of coronary artery disease (CAD): vulnerable plaque and stable plaque. While both plaque types can contribute to stenosis requiring invention consistent with the teachings of the present invention, vulnerable plaque is more frequently associated with sudden coronary death resulting from plaque rupture and the associated thrombogenic processes. Stable plague is not prone to rupture due to the presence of a thick fibrous cap and less amorphous, more stable, smaller lipid core than found in vulnerable plaque. Therefore, the majority of vascular stenoses requiring intervention are associated with stable plaque.

In one embodiment of the present invention percutaneous transluminal coronary angioplasty (PTCA), or balloon angioplasty, is used to correct stenoses found in coronary, iliac or kidney arteries. Moreover, the present invention PTCA is combined with stent deployment. Stents are mesh-like structures or coils that are mounted to an angioplasty balloon or on self-expanding devices and are permanently placed in the artery or vein following balloon expansion of the stricture.

In the typical procedure a patient is brought to the cardiac catheterization lab where intravenous fluids and medications are administered prior to beginning PTCA. Patients may also receive intravenous sedation to provide some comfort and anxiety relief. Next arterial and venous punctures are made and a sheath is inserted to provide access to the vascular system for a guidewire and coronary catheter. The coronary catheter is advanced over the guidewire and gently brought near the orifice of the coronary arteries. The guidewire is then removed and intravenous x-ray contrast dye is injected into the coronary arteries to facilitate visualizing the exact location of the stricture and the degree of narrowing. The patient's blood pressure, heart rate, electrocardiogram, and oxygen saturation are monitored continuously.

If severe stenosis of the coronary arteries is identified, an angioplasty balloon is inflated to dilate the stenosed region and a vascular stent is deployed to prevent immediate tissue elastic recoil and arterial re-occlusion. Exact stent placement is confirmed using repeat x-rays and when appropriate, intra-coronary ultrasound. One of the major complications associated with vascular stenting is restenosis. Restenosis results from injury to the vascular endothelium associated PTCA and stenting procedures. Briefly, the process of inflating the balloon catheter can tear the vessels' initmal layer of endothelial cells. The damaged endothelial cells secrete growth factors and other mitogenic agents causing monocytes and vascular smooth muscle cells (VSMCs) to migrate from the sub endothelium and into the arterial wall's intimal layer.

Other embodiments of the present invention include stenting procedures for peripheral vascular disease, such as, but not limited to, iliac artery stenosis, renal hypertension due to severe renal artery stenosis, and carotid artery stenosis. Moreover, neurovascular applications of the present invention are also considered within the scope of the present invention.

In the detailed description and claims that follows the compounds used to prevent restenosis may be referred to herein or elsewhere as imatinib mesylate, Gleevec™, Glivec™, Glivac, CGP 57148B, CGP 53716STI-571, protein-tyrosine kinase inhibitors, anti-restenotics, anti-restenotic compounds, drugs, therapeutics, anti-proliferatives, cytostatic agents, cytotoxic agents, or anti-metabolic agents. Gleevec™ and Glivec™ are trademarks of Novartis AG Corporation Switzerland Ch-4002 Basel Switzerland and referred to the chemical composition 4-[(4-Methyl-1-piperazinyl)methyl]-N-[4-methyl-3-[[4-(3-pyridinyl)-2-pyrimidinyl]amino]-phenyl]benzamide methanesulfonate (also known as imatinib mesylate). Presently, imatinib mesylate, in pharmaceutically acceptable forms, has been approved for treating chronic myeloid leukemia (CML). Furthermore, in the description and claims that follow, their trade name, chemical names or common names may refer to specific compounds. All of these terms may be used interchangeably without distinction and are all considered to within the scope of the present invention.

Neo-intima formation resulting from VSMC proliferation at the site of vascular injury accounts for the majoring of non-elastic recoil restenosis. Physical stress applied to the stenosed artery's intimal lining during angioplasty often results in rupture of the endothelial layer and damage to the underlying VSMC layer. The associated cell injury triggers a cascade of events that result in platelet aggregation and the paracrine and autocrine release of growth factors such as PDGF. The released growth factors then bind to receptors on VSMC causing the VSMCs to de-differentiate and proliferate through the damaged intima re-occluding the artery (See Myllärniemi, M. et al. 1999. Selective Tyrosine Kinase Inhibitor for the Platelet-Derived Growth Factor Receptor In Vitro Inhibits Smooth Muscle Cell Proliferation After Re-Injury of Arterial Intima In Vivo. Cardiovascular Drugs and Therapy; 13:159-168.)

Growth factor such as PDGF bind to specific receptors on cell surfaces and activate a family of intracellular phosphotransferase enzymes known as kinases. Kinases participate in the transfer of phosphate groups, usually from ATP, to specific molecular targets within a cell (phosphorylation). In the present case, the molecular target is a protein-tyrosine. Protein-tyrosine kinases phosphorylate proteins on their tyrosine residues using ATP as the phosphate donor. The phosphorylation of protein-tyrosine initiates a series of down-stream processes involved in the signaling process associated with cell growth. In its simplest conceptual form, the protein-tyrosine kinase receptor is a switch that must be turned on in order for a cell to proliferate. In the present case, the key that turns the switch on is PDGF. When PDGF binds to the protein-tyrosine kinase membrane receptor, phosporylation of protein-tyrosine results which in turn initiates biochemical pathways leading to cell proliferation. Imatinib mesylate mimics PDGF sufficiently to effectively block the protein-tyrosine membrane receptor keeping PDGF from switching on the cell proliferation pathway. Therefore, the present inventors believe that the in situ delivery of imatinib mesylate in amounts sufficient to block VSMC protein-tyrosine membrane receptors can prevent neo-intima formation and hence restenosis.

The present invention includes novel compositions and methods for delivering protein-tyrosine kinase inhibitors directly to tissues susceptible to restenosis. Specifically, the present invention is directed at implantable medical devices that provide for the in situ, site-specific controlled release of ligands that bind to and prevent activation of platelet-derived growth factor (PDGF) receptors inhibiting vascular smooth muscle cell (VSMC) proliferation.

In one embodiment of the present invention medical devices are provided with a protein-tyrosine kinase inhibitor such as, but not limited to 4-[(4-Methyl-1-piperazinyl)methyl]-N-[4-methyl-3-[[4-(3-pyridinyl)-2-pyrimidinyl]amino]-phenyl]benzamide methanesulfonate (imatinib mesylate) as depicted in Formula 1:

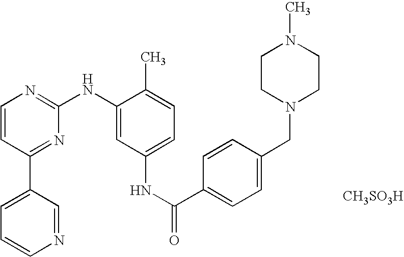

### 4-[(4-Methyl-1-piperazinyl)methyl]-N-[4-methyl-3-[[4-(3-pyridinyl)-2-pyrimidinyl]amino]-phenyl]benzamide methanesulfonate (imatinib mesylate or Gleevec™)

### Formula 1

It will be understood by those skilled in the art that Formula 1 is but one of many pharmaceutically acceptable salts of the protein-tyrosine kinase inhibitor of the present invention. Many other salts and other pharmaceutically acceptable forms can be synthesized and are still considered to within the scope of the present invention. Moreover, many derivatives are also possible that do not affect the efficacy or mechanism of action of the protein-tyrosine kinase inhibitor of the present invention. Therefore, the present invention is intended to encompass 4-[(4-Methyl-1-piperazinyl)methyl]-N-[4-methyl-3-[[4-(3-pyridinyl)-2-pyrimidinyl]amino]-phenyl]benzamide methanesulfonate (imatinib mesylate or Gleevec™) and pharmaceutically acceptable derivatives and salts thereof. The term “pharmaceutically acceptable” as used herein includes all derivatives and salts that are not substantially toxic at anti-restenotic effective levels in vivo. “Not substantially toxic” as used herein shall mean systemic or localized toxicity wherein the benefit to the recipient is out-weighed by the physiologically harmful effects of the treatment as determined by physicians and pharmacologists having ordinary skill in the art of chemotherapy and toxicology.

The protein-tyrosine kinase inhibitors of the present invention may be delivered, alone or in combination with synergistic and/or additive therapeutic agents, directly to the affected area using medical devices. Potentially synergistic and/or additive therapeutic agents may include drugs that impact a different aspect of the restenosis process such as antiplatelet, antimigratory or antifibrotic agents. Alternately they may include drugs that also act as antiproliferatives and/or anti-inflammatories. For example, and not intended as a limitation, synergistic combination, considered to within the scope of the present invention include at least one protein-tyrosine kinase inhibitor and bioactive compound is an anti-proliferative including, but not limited to, macrolide antibiotics including FKBP 12 binding compounds, estrogens, chaperone inhibitors, protein-tyrosine kinase inhibitors, peroxisome proliferator-activated receptor gamma ligands (PPARγ), hypothemycin, nitric oxide, bisphosphonates, epidermal growth factor inhibitors, antibodies, proteasome inhibitors, anti-sense nucleotides and transforming nucleic acids, at least one protein-tyrosine kinase inhibitor and rapamycin or analogues and derivatives thereof such a 40-O-(2-hydroxyethyl)-rapamycin or ABT-578 (Abbott Laboratories, see U.S. Pat. No. 6,015,815) at least one protein-tyrosine kinase inhibitor and exochelin, at least one protein-tyrosine kinase inhibitor and n-acetyl cysteine inhibitors, at least one protein-tyrosine kinase inhibitor and geldanamycin and other chaperone inhibitors, at least one protein-tyrosine kinase inhibitor and a PPARγ agonist and so on.

The medical devices used in accordance with the teachings of the present invention may be permanent medical implants, temporary implants, or removable devices. For example, and not intended as a limitation, the medical devices of the present invention may include, stents, catheters, micro-particles, probes and vascular grafts.

In one embodiment of the present invention stents are used as the drug delivery platform. The stents may be vascular stents, urethral stents, biliary stents, or stents intended for use in other ducts and organ lumens. Vascular stents may be used in peripheral, neurological or coronary applications. The stents may be rigid expandable stents or pliable self-expanding stents. Any biocompatible material may be used to fabricate or the stents of the present invention including, without limitation, metals or polymers. The stents of the present invention may also be bioresorbable. In one embodiment of the present invention vascular stents are implanted into coronary arteries immediately following angioplasty.

In one embodiment of the present invention metallic vascular stents are coated with one or more anti-restenotic compound, specifically protein-tyrosine kinase inhibitors, more specifically the protein-tyrosine kinase inhibitor is imatinib mescylate. The protein-tyrosine kinase inhibitor may be dissolved or suspended in any carrier compound that provides a stable composition that does not react adversely with the device to be coated or inactivate the protein-tyrosine kinase inhibitor. The metallic stent is provided with a biologically active protein-tyrosine kinase inhibitor coating using any technique known to those skilled in the art of medical device manufacturing. Suitable non-limiting examples include impregnation, spraying, brushing, dipping and rolling. After the protein-tyrosine kinase inhibitor-containing solution is applied to the stent it is dried leaving behind a stable protein-tyrosine kinase inhibitor delivering medical device. Drying techniques include, but are not limited to heated forced air, cooled forced air, vacuum drying or static evaporation. Moreover, the medical device, specifically a metallic vascular stent, can be fabricated having grooves or wells in its surface that serve as receptacles or reservoirs for the protein-tyrosine kinase inhibitor compositions of the present invention.

The preferred concentration of protein-tyrosine kinase inhibitor used in accordance with the teachings of the present invention can be determined using a titration process. Titration is accomplished by preparing a series of stent sets. Each stent set will be coated, or contain different dosages of the protein-tyrosine kinase inhibitor selected. The highest concentration used will be partially based on the known toxicology of the compound. The maximum amount of drug delivered by the stents made in accordance with the teaching of the present invention will fall below known toxic levels. Each stent set will be tested in vivo using the preferred animal model as described in Example 5 below. The dosage selected for further studies will be the minimum dose required to achieve the desired clinical outcome. In the case of the present invention, the desired clinical outcome is defined as the inhibition of vascular re-occlusion, or restenosis.

In another embodiment the protein-tyrosine kinase inhibitor is precipitated or crystallized on or within the stent. In yet another embodiment the protein-tyrosine kinase inhibitor is mixed with a suitable biocompatible polymer (bioerodable, bioresorbable or non-erodable). The polymer-protein-tyrosine kinase inhibitor blend can then be used to produce a medical device such as, but not limited to stents, grafts, micro-particles, sutures and probes. Furthermore, the polymer-protein tyrosine kinase inhibitor blend can be used to form a controlled release coating for the medical device surfaces. For example, and not intended as a limitation, the medical device can be immersed in the polymer-protein tyrosine kinase inhibitor blend, or the polymer-protein-tyrosine kinase inhibitor blend can be sprayed, or brushed onto the medical device. In another embodiment, the polymer-protein-tyrosine kinase inhibitor blend can be used to fabricate fibers or strands that are embedded into the medical device or used to wrap the medical device.

Polymers used in accordance with the teachings of the present invention include both bio-absorbable and non-absorbable materials. Any combination of polymers that exhibit the properties of biocompatibility, high elasticity/ductility, resistance to erosion (except in the case of bio-erodable/absorbable polymers), elasticity, and controlled drug release are within the scope of the present invention and may be used to form the medical devices, or preferably controlled release device coatings. Suitable non-limiting exemplary monomers include hydroxy alkyl methacrylate, N-vinyl pyrrolidine, alkyl methacrylate, vinyl alcohols, acrylic acids, acrylamides, ethylene, vinyl acetate, ethylene glycol, methacrylic acid, phosphorylcholine, caprolactone, lactic acid and co-polymers thereof.

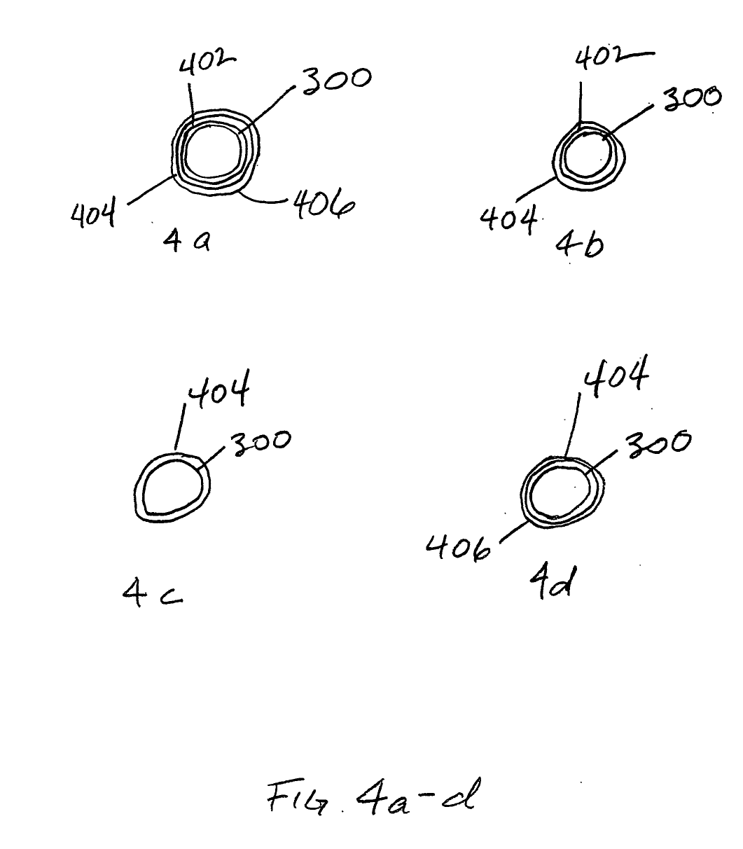

Several exemplary embodiments of the present invention are depicted in FIG. 3. In FIG. 3 a vascular stent 300 having the structure 302 is made from a material selected from the non-limiting group materials including stainless steel, nitinol, aluminum, chromium, titanium, ceramics, and a wide range of plastics and natural materials including collagen, fibrin and plant fibers. The structure 302 is provided with a coating composition made in accordance with the teachings of the present invention. FIG. 4a-d are cross-sections of stent 300 showing various coating configurations. In FIG. 4a stent 300 has a first polymer coating 402 comprising a medical grade primer, such as but not limited to parylene or a parylene derivative; a second controlled release coating 404; and a third barrier, or cap, coat 406. In FIG. 4b stent 300 has a first polymer coating 402 comprising a medical grade primer, such as but not limited to parylene or a parylene derivative and a second controlled release coating 404. In FIG. 4c stent 300 has a first controlled release coating 404 and a second barrier, or cap, coat 406. In FIG. 4d stent 300 has only a controlled release coating 404.

The following examples are provided to more precisely define and enable the protein-tyrosine kinase inhibitor-eluting medical devices of the present invention. It is understood that there are numerous other embodiments and methods of using the present invention that will be apparent to those of ordinary skill in the art after having read and understood this specification and examples. These alternate embodiments are considered part of the present invention.

### EXAMPLES

### Providing a Metallic Surface with a Protein-Tyrosine Kinase-Eluting Coating

The following Examples are intended to illustrate a non-limiting process for coating metallic stents with a protein-tyrosine kinase inhibitor and testing their anti-restenotic properties. One non-limiting example of a metallic stent suitable for use in accordance with the teachings of the present invention is the Medtronic/AVE S670™ 316L stainless steel coronary stent.

### Example 1

### Metal Stent Cleaning Procedure

Stainless steel stents were placed a glass beaker and covered with reagent grade or better hexane. The beaker containing the hexane immersed stents was then placed into an ultrasonic water bath and treated for 15 minutes at a frequency of between approximately 25 to 50 KHz. Next the stents were removed from the hexane and the hexane was discarded. The stents were then immersed in reagent grade or better 2-propanol and vessel containing the stents and the 2-propanol was treated in an ultrasonic water bath as before. Following cleaning the stents with organic solvents, they were thoroughly washed with distilled water and thereafter immersed in 1.0 N sodium hydroxide solution and treated at in an ultrasonic water bath as before. Finally, the stents were removed from the sodium hydroxide, thoroughly rinsed in distilled water and then dried in a vacuum oven over night at 40° C.

After cooling the dried stents to room temperature in a desiccated environment they were weighed their weights were recorded.

### Example 2

### Coating a Clean, Dried Stent Using a Drug/Polymer System

In the following Example, methanol is chosen as the solvent of choice. Both the polymer and imatinib mesylate are freely soluble ion methanol. Imatinib mesylate is also known to be freely soluble in water, slightly acidic buffered aqueous solutions, dimethyl sulfoxide, methanol, and ethanol. Imatinib mesylate is insoluble in neutral and alkaline aqueous solutions, n-octanol, acetone and acetonitrile. Persons having ordinary skill in the art of polymer chemistry can easily pair the appropriate solvent system to the polymer-drug combination and achieve optimum results with no more than routine experimentation.

250 mg of imatinib mesylate is carefully weighed and added to a small neck glass bottle containing 2.8 ml of methanol. The imatinib mesylate-methanol suspension is then thoroughly mixed until a clear solution is achieved.

Next 250 mg of polycaprolactone (PCL) is added to the imatinib mesylate-methanol solution and mixed until the PCL dissolved forming a drug/polymer solution.

The cleaned, dried stents are coated using either spraying techniques or dipped into the drug/polymer solution. The stents are coated as necessary to achieve a final coating weight of between approximately 10 μg to 1 mg. Finally, the coated stents are dried in a vacuum oven at 50° C. over night. The dried, coated stents are weighed and the weights recorded.

The concentration of drug loaded onto (into) the stents is determined based on the final coating weight. Final coating weight is calculated by subtracting the stent's pre-coating weight from the weight of the dried, coated stent.

### Example 3

### Coating a Clean, Dried Stent Using a Sandwich-type Coating

A cleaned, dry stent is first coated with polyvinyl pyrrolidone (PVP) or another suitable polymer followed by a coating of imatinib mesylate. Finally, a second coating of PVP is provided to seal the stent thus creating a PVP-imatinib mesylate-PVP sandwich coated stent.

The Sandwich Coating Procedure:

100 mg of PVP is added to a 50 mL Erlenmeyer containing 12.5 ml of methanol. The flask was carefully mixed until all of the PVP is dissolved. In a separate clean, dry Erlenmeyer flask 250 mg of imatinib mesylate is added to 11 mL of methanol and mixed until dissolved.

A clean, dried stent is then sprayed with PVP until a smooth confluent polymer layer was achieved. The stent was then dried in a vacuum oven at 50° C. for 30 minutes.

Next, successive layers of imatinib mesylate are applied to the polymer-coated stent. The stent is allowed to dry between each of the successive imatinib mesylate coats. After the final imatinib mesylate coating has dried, three successive coats of PVP are applied to the stent followed by drying the coated stent in a vacuum oven at 50° C. over night. The dried, coated stent is weighed and its weight recorded.

The concentration of drug in the drug/polymer solution and the final amount of drug loaded onto the stent determine the final coating weight. Final coating weight is calculated by subtracting the stent's pre-coating weight from the weight of the dried, coated stent.

### Example 4

### Coating a Clean, Dried Stent with Pure Drug

1.00 g of imatinib mesylate is carefully weighed and added to a small neck glass bottle containing 12 ml of methanol. The imatinib mesylate-methanol suspension is then heated at 50° C. for 15 minutes and then mixed until the imatinib mesylate is completely dissolved.

Next a clean, dried stent is mounted over the balloon portion of angioplasty balloon catheter assembly. The stent is then sprayed with, or in an alternative embodiment, dipped into, the imatinib mesylate-methanol solution. The coated stent is dried in a vacuum oven at 50° C. over night. The dried, coated stent was weighed and its weight recorded.

The concentration of drug loaded onto (into) the stents is determined based on the final coating weight. Final coating weight is calculated by subtracting the stent's pre-coating weight from the weight of the dried, coated stent.

### Example 5

### In Vivo Testing of a protein-Tyrosine Kinase Inhibitor-Coated Vascular Stent in a Porcine Model

The ability of a protein-tyrosine kinase inhibitor to reduce neointimal hyperplasia in response to intravascular stent placement in an acutely injured porcine coronary artery is demonstrated in the following example. Two controls and three treatment arms were used as outlined below:

1. Control Groups:
Six animals were used in each control group. The first control group tests the anti-restenotic effects of the clean, dried stent having neither polymer nor drug coatings. The second control group tests the anti-restenotic effects of polymer alone. Clean, dried stents having PCL coatings without drug are used in the second control group.

2. Experimental Treatment Groups
Three different stent configurations and two different drug dosages are evaluated for their anti-restenotic effects. Twelve animals are included in each group.

Group 1 stents are designated the fast release group and are comprised of 50 μg imatinib mesylate coated onto a bare stent without polymer in accordance with the teachings of the present invention.

Group 2, designated the slow-release group, uses stents coated with 50 μg of imatinib mesylate impregnated within a polymer at an imatinib mesylate to polymer ratio of 1:9 in accordance with the teachings of the present invention.

Group 3, designated the medium-release group, uses stents coated with 250 μg of imatinib mesylate impregnated within a polymer at an imatinib mesylate to polymer ratio of 1:1 in accordance with the teachings of the present invention.

The swine has emerged as the most appropriate model for the study of the endovascular devices. The anatomy and size of the coronary vessels are comparable to that of humans. Furthermore, the neointimal hyperplasia that occurs in response to vascular injury is similar to that seen clinically in humans. Results obtained in the swine animal model are considered predictive of clinical outcomes in humans. Consequently, regulatory agencies have deemed six-month data in the porcine sufficient to allow progression to human trials.

Non-atherosclerotic acutely injured RCA, LAD, and/or LCX arteries of the Farm Swine (or miniswine) are utilized in this study. Placement of coated and control stents is random by animal and by artery. The animals are handled and maintained in accordance with the requirements of the Laboratory Animal Welfare Act (P.L.89-544) and its 1970 (P.L. 91-579), 1976 (P.L. 94-279), and 1985 (P.L. 99-198) amendments. Compliance is accomplished by conforming to the standards in the Guide for the Care and the Use of Laboratory Animals, ILAR, National Academy Press, revised 1996. A veterinarian performs a physical examination on each animal during the pre-test period to ensure that only healthy pigs are used in this study.

A. Pre-Operative Procedures

The animals are monitored and observed 3 to 5 days prior to experimental use. The animals have their weight estimated at least 3 days prior to the procedure in order to provide appropriate drug dose adjustments for body weight. At least one day before stent placement, 650 mg of aspirin is administered. Animals are fasted twelve hours prior to the procedure.

B. Anesthesia

Anesthesia is induced in the animal using intramuscular Telazol and Xylazine. Atropine is administered (20 μg/kg I.M.) to control respiratory and salivary secretions. Upon induction of light anesthesia, the subject animal is intubated. Isoflurane (0.1 to 5.0% to effect by inhalation) in oxygen is administered to maintain a surgical plane of anesthesia. Continuous electrocardiographic monitoring is performed. An I.V. catheter is placed in the ear vein in case it is necessary to replace lost blood volume. The level of anesthesia is monitored continuously by ECG and the animal's response to stimuli.

C. Catheterization and Stent Placement

Following induction of anesthesia, the surgical access site is shaved and scrubbed with chlorohexidine soap. An incision is made in the region of the right or left femoral (or carotid) artery and betadine solution is applied to the surgical site. An arterial sheath is introduced via an arterial stick or cutdown and the sheath is advanced into the artery. A guiding-catheter is placed into the sheath and advanced via a 0.035″ guide wire as needed under fluoroscopic guidance into the ostium of the coronary arteries. An arterial blood sample is obtained for baseline blood gas, ACT and HCT. Heparin (200 units/kg) is administered as needed to achieve and maintain ACT ≧300 seconds. Arterial blood pressure, heart rate, and ECG are recorded.

After placement of the guide catheter into the ostium of the appropriate coronary artery, angiographic images of the vessels are obtained in at least two orthagonal views to identify the proper location for the deployment site. Quantitative coronary angiography (QCA) is performed and recorded. Nitroglycerin (200 μg I.C.) is administered prior to treatment and as needed to control arterial vasospasm. The delivery system is prepped by aspirating the balloon with negative pressure for five seconds and by flushing the guidewire lumen with heparinized saline solution.

Deployment, patency and positioning of stent are assessed by angiography and a TIMI score is recorded. Results are recorded on video and cine. Final lumen dimensions are measured with QCA and/or IVUS. These procedures are repeated until a device is implanted in each of the three major coronary arteries of the pig. After final implant, the animal is allowed to recover from anesthesia. Aspirin is administered at 325 mg p.o. qd until sacrifice.

D. Follow-Up Procedures and Termination

After 28 days, the animals are anesthetized and a 6F arterial sheath is introduced and advanced. A 6F large lumen guiding-catheter (diagnostic guide) is placed into the sheath and advanced over a guide wire under fluoroscopic guidance into the coronary arteries. After placement of the guide catheter into the appropriate coronary ostium, angiographic images of the vessel are taken to evaluate the stented sites. At the end of the re-look procedure, the animal is euthanized with an overdose of Pentabarbitol I.V. and KCL I.V. The heart, kidneys, and liver are harvested and visually examined for any external or internal trauma. The organs are flushed with 1000 ml of lactated ringers at 100 mmHg and then flushed with 1000 ml of formalin at 100-120 mmHg. All organs are stored in labeled containers of formalin solution.

E. Histology and Pathology

The stented vessels will be X-rayed prior to histology processing. The stented segments are processed for routine histology, sectioned, and stained following standard histology lab protocols. Appropriate stains are applied in alternate fashion on serial sections through the length of the treated vessels.

F. Data Analysis and Statistics

1. QCA Measurement

Quantitative angiography is performed to measure the balloon size at peak inflation as well as vessel diameter pre- and post-stent placement and at the 28 day follow-up. The following data are measured or calculated from angiographic data:
Stent-to-artery-ratio
Minimum lumen diameter (MLD)
Distal and proximal reference lumen diameter
   Percent Stenosis=(Minimum lumen diameter÷reference lumen diameter)×100

2. Histomorphometric Analysis

Histologic measurements are made from sections from the native proximal and distal vessel and proximal, middle, and distal portions of the stent. A vessel injury score is calculated using the method described by Schwartz et al. (Schwartz RS et al. Restenosis and the proportional neointimal response to coronary artery injury: results in a porcine model. J Am Coll Cardiol 1992; 19:267-74). The mean injury score for each arterial segment is calculated. Investigators scoring arterial segment and performing histopathology are “blinded” to the device type. The following measurements are determined:
External elastic lamina (EEL) area
Internal elastic lamina (IEL) area
Luminal area
Adventitial area
Mean neointimal thickness
Mean injury score

3. The Neointimal Area and the % of In-Stent Restenosis are Calculated as Follows:
Neointimal area=(IEL-luminal area)
In-stent restenosis=[1-(luminal area÷IEL)]×100.

A given treatment arm will be deemed beneficial if treatment results in a significant reduction in neointimal area and/or in-stent restenosis compared to both the bone stent control and the polymer-on control.

G. Surgical Supplies and Equipment

The following surgical supplies and equipment are required for the procedures described above:
1. Standard vascular access surgical tray
2. Non-ionic contrast solution
3. ACT machine and accessories
4. HCT machine and accessories (if applicable)
5. Respiratory and hemodynamic monitoring system
6. IPPB Ventilator, associated breathing circuits and Gas Anesthesia Machine
7. Blood gas analysis equipment
8. 0.035″ HTF or Wholey modified J guidewire, 0.014″ Guidewires
9. 6, 7, 8, and 9F introducer sheaths and guiding catheters (as applicable)
10. Cineangiography equipment with QCA capabilities
11. Ambulatory defibrillator
12. Standard angioplasty equipment and accessories
13. IVUS equipment (if applicable)
14. For radioactive labeled cell studies (if applicable):
15. Centrifuge
16. Aggregometer
17. Indium 111 oxime or other as specified
18. Automated Platelet Counter
19. Radiation Detection Device

### Example 6

### Inhibition of Human Coronary Artery Smooth Muscle Cells by Imatinib Mesylate

A. Materials
1. Human coronary smooth muscles cells (HCASMC) are obtained from Clonetics, a division of Cambrex, Inc.
2. HCASMC basal media is supplied by Clonetics and is supplemented with fetal bovine serum, insulin, hFGF-B (human fibroblast growth factor) hEGF (human epidermal growth factor).
3. Imatinib Mesylate (Gleevec™: Novartis AG Corporation Switzerland Ch-4002 Basel Switzerland)
4. Absolute Ethanol
5. Twenty-four well polystyrene tissue culture plates

B. Human Coronary Artery Smooth Muscle Cells Proliferation Inhibition Studies. Imatinib mesylate

Human coronary smooth muscles cells (HCASMC) are seeded in 24 well polystyrene tissue culture plates at a density of 5×10³ cells per well. Two different feeding and reading strategies are employed. Strategy 1: Cells are plated in cell culture media containing various concentrations of imatinib mesylate and incubated at 37° C. for 48 hours. After the initial 48 hour incubation, the imatinib mesylate containing plating media is changed and the cells are fed with drug free media and incubated for an additional 48 hours and then read.

Strategy 2: Cells are plated in cell culture media containing various concentrations of imatinib mesylate and incubated at 37° C. for 48 hours. After the initial 48 hour incubation, the imatinib mesylate-containing plating media is changed and the cells are fed with imatinib mesylate-containing media and incubated for an additional 48 hours and then read.

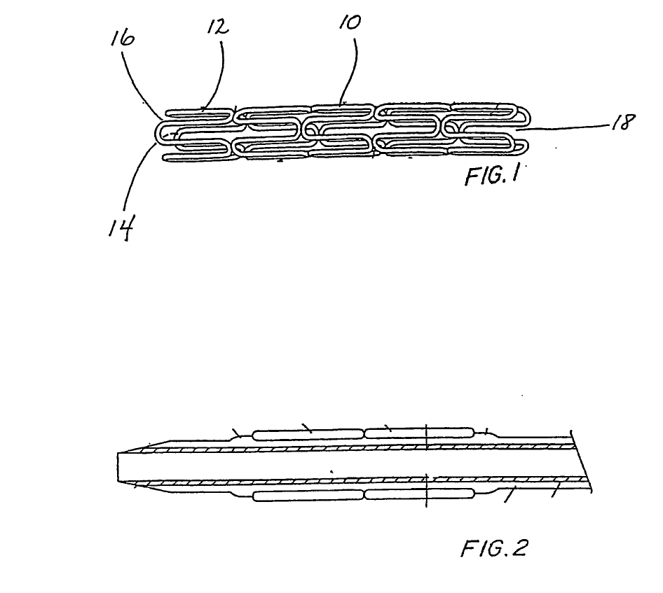

On day four cultures are analyzed to determine the proliferation inhibition effects of imatinib mesylate. FIG. 1a graphically depicts the percent inhibition at imatinib mesylate levels between 0.001 μg/mL to 50 μg/mL for both cell culture schemes. It can be seen from FIG 1a that significant HCASMC inhibition (>50% inhibition) begins at a dosage of 10 μg/mL and rises dramatically to nearly 100% at 50 μg/mL. FIG. 1b graphically depicts the same results in bar graph form based on cell counts.

C. Imatinib Mesylate Cytotoxicity Testing

Imatinib mesylate cytotoxicity against HCASMCs is evaluated by seeding 24 well cell culture plates with 5.0×10⁵ HCASM cells/mL of cell culture media containing from 0.001 μg/mL to 10 μg/mL of imatinib mesylate. Samples are taken after 24 hours and tested for lactate dehydrogenase (LDL) concentration using methods known to those having ordinary skill in the art. Elevated LDL levels indicates cytotoxicity. FIG. 1c graphically depicts the cytotoxicity testing results. No cytotoxicity is detected at imatinib mesylate concentrations that demonstrated significant anti-proliferative effects.

### Example 7

### Inhibition of Human Coronary Artery Endothelial Cells by Imatinib Mesylate

A. Materials
1. Human coronary artery endothelial cells (HCAEC) are obtained from Clonetics, a division of Cambrex, Inc.
2. HCAEC basal media is supplied by Clonetics and is supplemented with fetal bovine serum, VEGF (vascular endothelial growth factor) hEGF heparin, ascorbic acid IGF (insulin growth factor) hydrocortisone
3. Imatinib Mesylate (Gleevec™: Novartis AG Corporation Switzerland Ch-4002 Basel Switzerland)
4. Absolute Ethanol
5. Twenty-four well polystyrene tissue culture plates
   B. Human Coronary Smooth Muscles Cells Proliferation Inhibition Studies.

Human coronary artery endothelial cells (HCAEC) are seeded in 24 well polystyrene tissue culture plates at a density of 5×10³ cells per well. Two different feeding strategies and reading strategies are employed. Strategy 1: Cells are plated in cell culture media containing various concentrations of imatinib mesylate and incubated at 37° C. for 48 hours. After the initial 48 hour incubation, the imatinib mesylate containing plating media is changed and the cells are fed with drug free media and incubated for an additional 48 hours and then read.

Strategy 2: Cells are plated in cell culture media containing various concentrations of imatinib mesylate and incubated at 37° C. for 48 hours. After the initial 48 hour incubation, the imatinib mesylate-containing plating media is changed and the cells are fed with imatinib mesylate-containing media and incubated for an additional 48 hours and then read.

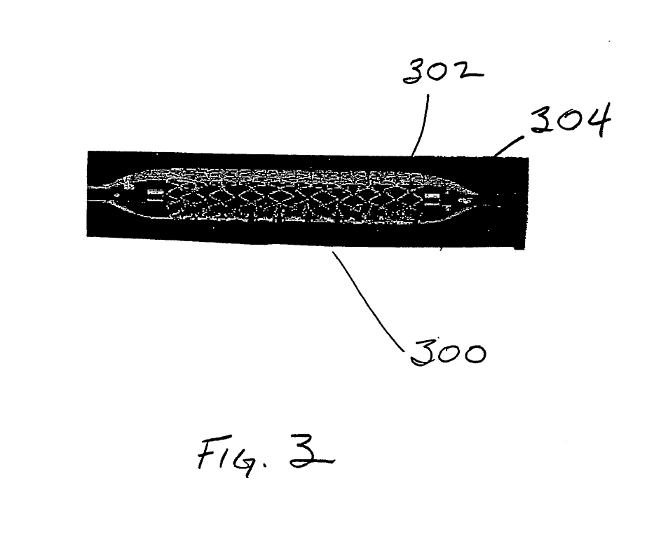

On day four cultures are analyzed to determine the proliferation inhibition effects of imatinib mesylate. FIG. 1 a graphically depicts the percent inhibition at imatinib mesylate levels between 0.001 μg/mL to 50 μg/mL for both cell culture schemes. It can be seen from FIG. 2a that significant HCAEC inhibition (>50% inhibition) begins at a dosage of 5 μg/mL and rises dramatically to nearly 100% at 10 μg/mL. FIG. 2b graphically depicts the same results in bar graph form based on cell counts.

C. Imatinib Mesylate Cytotoxicity Testing

Imatinib mesylate cytotoxicity against HCAECs is evaluated by seeding 24 well cell culture plates with 5.0×10⁵ HCAE cells/mL of cell culture media containing from 0.001 μg/mL to 10 μg/mL of imatinib mesylate. Samples are taken after 24 hours and tested for lactate dehydrogenase (LDL) concentration using methods known to those having ordinary skill in the art. Elevated LDL levels indicate cytotoxicity. FIG. 2c graphically depicts the cytotoxicity testing results. No cytotoxicity is detected at imatinib mesylate concentrations that demonstrated significant anti-proliferative effects.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A medical device comprising an implantable device for the site-specific, controlled release delivery of a therapeutic amount of a protein-tyrosine kinase inhibitor.

2. The medical device according to claim 1 wherein said protein-tyrosine kinase inhibitor has the molecular structure according to Formula 1; 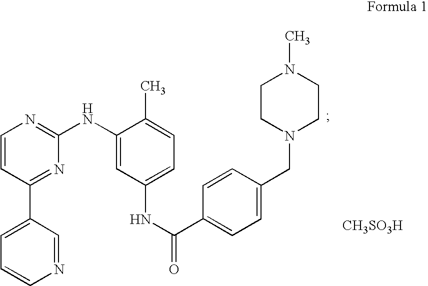
and pharmaceutically acceptable derivatives thereof.

3. The medical device according to claim 2 wherein said protein-tyrosine kinase inhibitor is 4-[(4-Methyl-1-piperazinyl)methyl]-N-[4-methyl-3-[[4-(3-pyridinyl)-2-pyrimidinyl]amino]-phenyl]benzamide methanesulfonate (imatinib mesylate) and pharmaceutically acceptable derivatives thereof.

4. The medical device according to any of claims 2 or 3 wherein said medical device is selected from the group consisting of stents, catheters, micro-particles, probes and vascular grafts.

5. The medical device according to claim 4 wherein said stent is a vascular stent, esophageal stent, urethral stent or biliary stent.

6. The medical device according to claim 5 wherein said vascular stent is provided with a coating comprising imatinib mesylate and pharmaceutically acceptable derivatives thereof.

7. The medical device according to claim 6 wherein said coating further contains a biocompatible polymer.

8. A vascular stent having a coating comprising imatinib mesylate and pharmaceutically acceptable derivatives thereof.

9. A medical device comprising a stent having a coating comprising imatinib mesylate and pharmaceutically acceptable derivatives thereof; and
a polymer.

10. The medical device according to claim 9 wherein said coating comprises:
between approximately 50 μg to 250 μg of a protein-tyrosine kinase inhibitor and a polymer wherein said protein-tyrosine kinase inhibitor and said polymer are in a ratio relative to each other of approximately 1 part protein-tyrosine kinase inhibitor to approximately between 1 to 9 parts polymer;
wherein said protein-tyrosine kinase inhibitor comprises imatinib mesylate and pharmaceutically acceptable derivatives thereof.

11. A method of treating or inhibiting restenosis comprising:
providing a vascular stent having a coating comprising a protein-tyrosine kinase inhibitor; and
implanting said vascular stent into a blood vessel lumen wherein said protein-tyrosine kinase inhibitor is released into tissue adjacent said blood vessel lumen;
wherein said protein-tyrosine kinase inhibitor comprises imatinib mesylate and pharmaceutically acceptable derivatives thereof.

12. The method according to claim 10 wherein said coating comprises:
between approximately 50 μg to 250 μg of a protein-tyrosine kinase inhibitor and a polymer wherein said protein-tyrosine kinase inhibitor and said polymer are in a ratio relative to each other of approximately 1 part protein-tyrosine kinase inhibitor to approximately between 1 to 9 parts polymer;
wherein said protein-tyrosine kinase inhibitor comprises imatinib mesylate and pharmaceutically acceptable derivatives thereof.

13. A method for producing a medical device comprising:
providing medical device to be coated;
compounding imatinib mesylate and pharmaceutically acceptable derivatives thereof with a carrier compound; and
coating said medical devices with said imatinib mesylate and pharmaceutically acceptable derivatives thereof compounded with said carrier compound.

14. The method according to claim 13 wherein said medical device is a vascular stent.

15. The method according to claim 13 further wherein said carrier compound is a biocompatible polymer.

16. A medical device comprising a stent having a coating comprising imatinib mesylate and pharmaceutically acceptable derivatives thereof; and
at least one additional therapeutic agent selected from the group consisting of antiplatelet agents, antimigratory agent, antifibrotic agents, antiproliferatives, antiinflammatories and combinations thereof providing that said additional therapeutic agent.

17. The medical device according to claim 16 wherein said at least one additional therapeutic agent is selected from the group consisting of antisense oligonucleotides, rapamycin, analogues of rapamycin, exochelin, n-acetyl cysteine inhibitors, chaperone inhibitors, PPAR-agonists and combinations thereof.

18. The medical device according to claim 17 wherein said antisense oligonucleotide is an anti-c-myc oligonucleotide.

19. The medical device according to claim 17 wherein said chaperone inhibitor is geldanamycin.

20. The medical device according to claim 17 wherein said rapamycin derivative is 40-O-(2-hydroxyethyl)-rapamycin or ABT-578.

21. A method of treating or inhibiting restenosis comprising:
providing a vascular stent having a coating comprising imatinib mesylate and pharmaceutically acceptable derivatives thereof and at least one additional therapeutic agent selected from the group consisting of antiplatelet agents, antimigratory agent, antifibrotic agents, antiproliferatives, antiinflammatories and combinations thereof; and
implanting said vascular stent into a blood vessel lumen wherein said imatinib mesylate and pharmaceutically acceptable derivatives thereof and at least one additional therapeutic agent are released into tissue adjacent to said blood vessel lumen.
